(19) United States
(12) Patent Application Publication
(10) Pub. No.: US 2017/0333388 A1
(43) Pub. Date: Nov. 23, 2017

(54) Therapeutic compound and its application in repairing diabetes-related cardiac injuries

(71) Applicant: Numen Biotech Co., Ltd, Taipei City (TW)

(72) Inventors: Shaw-Yih Liou, Taipei City (TW); Chih-Yang Huang, Taichung City (TW); Tsai-Jui Liou, Taipei City (TW); Tung-Sheng CHEN, Taichung City (TW); I-Te Liou, Taipei City (TW)

(21) Appl. No.: 15/160,519

(22) Filed: May 20, 2016

(51) Int. Cl. A61K 31/353; A61K 35/28; A61K 9/00; A61K 36/82; C12N 5/0775

(52) U.S. Cl. CPC A61K 31/353; A61K 36/82; C12N 5/0667; A61K 9/0019; A61K 35/28; C12N 2500/76

(57) ABSTRACT

The present invention is related to a therapeutic compound and its application in repairing diabetes-related cardiac injuries, particularly with providing a therapeutic composition containing the epigallocatechin gallate (EGCG) of green tea and the adipose-derived stem cells. The epigallocatechin gallate (EGCG) of green tea can enhance the ability of the adipose-derived stem cells to repaired damaged tissue. And the application is used for repairing the diabetes-related cardiac injuries.

### FIELD OF THE INVENTION

The present invention is related to a therapeutic compound and its application in repairing diabetes-related cardiac injuries, particularly with providing a therapeutic composition which contains the epigallocatechin gallate (EGCG) of green tea and a adipose-derived stem cells. The epigallocatechin gallate (EGCG) of green tea can enhance the ability of the adipose-derived stem cells for repairing damaged tissue. And the application is used for repairing the diabetes-related cardiac injuries.

### BACKGROUND OF THE INVENTION

Diabetes Mellitus (DM) is caused by lack of insulin or abnormal function of insulin on the target cell, so as to induce the sugar, protein and fat metabolism disorder. Some diseases are accompanied with diabetes clinically and these diseases are called diabetes complications, such as cardiovascular disease, kidney disease, peripheral vascular disease, eye disease, liver disease, or neuropathy or peripheral neuropathy and other diseases. Statistic data shows that two of every four diabetes patients would have cardiac dysfunction, which represents the heart disease is the major complication of diabetes.

Some studies indicates that the diabetes induced heart injury is through the blood glucose or the advanced glycation end products (AGEs). No matter what kind of stimulus, the oxidative stress in myocardial cells would be increased, and the increased oxidative stress would destroy the mitochondria in the cardiomyocytes, so as to increase the expression level of apoptosis-related protein (such as caspase-3 and t-Bad). In contrast, the decrease of cell survival proteins, such as p-Akt would cause pathological reactions in cardiomyocytes, such as apoptosis, cardiomyocyte hypertrophy, and fibrosis or inflammation reaction. These pathological reactions would cause cardiac function disorder, and the damaged cells would not be able to autologous regenerate after cardiomyocytes inflammation. Current medications cannot make damaged cells self-regenerate, and therefore cannot make the heart function recover neither can solve the problem of high blood glucose.

Stem cell therapy for treating heart and diabetes-induced cardiovascular disease, can make the damaged cell regenerate and restore the heart function. However, the researchers found that stem cells would have poor regeneration ability under high glucose concentration, thus, how to maintain the autologous regeneration ability of stem cells the in high glucose to repair diabetes-induced cardiac injuries is an urgent problem.

### SUMMARY OF THE INVENTION

Accordingly, one object of the present invention is to provide a therapeutic compound for repairing cardiac injuries, wherein the therapeutic compound comprises a stem cell.

Preferably, the cardiac injuries are caused by diabetes or high-blood glucose.

Preferably, the compound further comprises an epigallocatechin gallate (EGCG) of green tea.

Preferably, the stem cell is adipose-derived stem cell.

Preferably, the adipose-derived stem cells and the epigallocatechin gallate (EGCG) of green tea are pretreated for 2 hours.

Preferably, the concentration of the epigallocatechin gallate (EGCG) of green tea used for pre-treating the adipose-derived stem cell is lower than 20 μM.

Preferably, the concentration of the epigallocatechin gallate (EGCG) of green tea used for pre-treating the adipose-derived stem cell is 5-15 μg/mL.

Another object of the present invention is to provide a method for repairing cardiac injuries, which includes administrating a stem cell containing therapeutic compound into a subject via intravenous injection.

Preferably, the cardiac injuries are caused by diabetes or high-blood glucose.

Preferably, the stem cell is adipose-derived stem cell.

The therapeutic compound of claim 7, wherein the method includes administrating an epigallocatechin gallate (EGCG) of green tea pre-treated 1×10⁵ adipose-derived stem cells into a subject via intravenous injection.

Preferably, the adipose-derived stem cell and the epigallocatechin gallate (EGCG) of green tea are pretreated for 2 hours.

Preferably, the concentration of the epigallocatechin gallate (EGCG) of green tea used for pre-treating the adipose-derived stem cells is lower than 20 μg/mL.

Preferably, the concentration of the epigallocatechin gallate (EGCG) of green tea used for pre-treating the adipose-derived stem cells is 5-15 μM.

Another object of the present invention is to provide a method for increasing the carbohydrate tolerance and moving ability of stem cells, wherein the method includes adding an epigallocatechin gallate (EGCG) of green tea into an adipose-derived stem cell culture medium.

Preferably, the concentration of the epigallocatechin gallate (EGCG) of green tea is 5-15 μM.

Preferably, the method further comprises taking out the adipose-derived stem cells from the epigallocatechin gallate (EGCG) of green tea containing adipose-derived stem cells culture medium after 2 hours culture.

### BRIEF DESCRIPTION OF THE DRAWINGS

FIG. 1 illustrates the features of the adipose-derived stem cells by identifying the positive markers and negative markers of the adipose-derived stem cells at passage 2;

FIG. 2 shows test result of differentiation ability of the adipose-derived stem cells (ADSC) at passage 2;

FIG. 3 shows the adipose-derived stem cells (ADSC) colonies distribution, which represents the epigallocatechin gallate (EGCG) enhances adipose-derived stem cells capability in medium containing high concentration glucose (33 mM) through CXCR4 expression;

FIG. 4 shows the transwell migration assay of the adipose-derived stem cells (ADSC), which represents the epigallocatechin gallate (EGCG) precondition enhances adipose-derived stem cells (ADSC) migration in medium containing high concentration glucose (33 mM) through CXCR4 expression;

FIG. 5 shows the Western blot analysis for protein expression amount in ADSC preconditions with different dosages of epigallocatechin gallate (EGCG);

FIG. 6 shows the Western blot analysis for protein expression amount in adipose-derived stem cells (ADSC) with and without epigallocatechin gallate (EGCG) precondition in the presence of CXCR4 siRNA;

FIG. 7 shows the transwell migration assay of adipose-derived stem cells (ADSC), which represents the migration ability of adipose-derived stem cells (ADSC) with or without epigallocatechin gallate (EGCG) precondition in the presence or absence of CXCR4 siRNA;

FIG. 8 shows the Western blot analysis for protein expression amount in H9c2 cardiomyoblasts co-culture with adipose-derived stem cells (ADSC) or ADSC preconditioned with 10 μM epigallocatechin gallate (EGCG);

FIG. 9 shows the Western blot analysis for protein expression amount in H9c2 cardiomyoblasts co-culture with adipose-derived stem cells (ADSC) or ADSC preconditioned with 10 μM epigallocatechin gallate (EGCG) in the presence of high concentration glucose (33 mM);

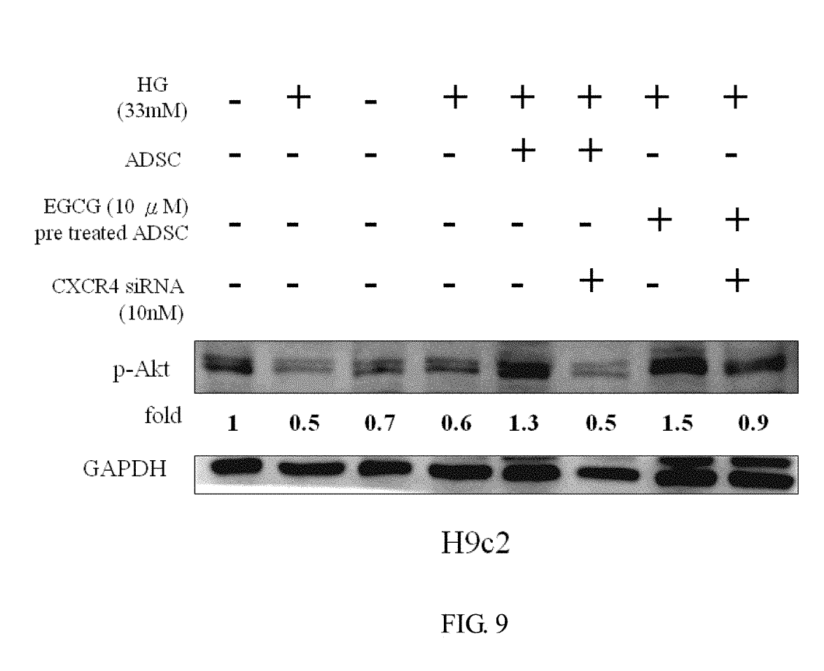

FIG. 10 illustrates change of serum glucose level and body weight in DM rats with autologous transplantation of adipose-derived stem cells (ADSC) with and without epigallocatechin gallate (EGCG) precondition;

FIG. 11 illustrates echocardiography and cardiac function in DM rats with autologous transplantation of adipose-derived stem cells (ADSC) with and without epigallocatechin gallate (EGCG) precondition;

FIG. 12 illustrates a cardiac blood ejection fraction (EF %) and fractional shortening (FS %) of DM rats with autologous transplantation of adipose-derived stem cells (ADSC) with and without epigallocatechin gallate (EGCG) precondition;

FIG. 13 illustrates cardiac histological change in DM rats with autologous transplantation of adipose-derived stem cells (ADSC) with and without epigallocatechin gallate (EGCG) precondition;

FIG. 14 shows the investigation of cardiac survival protein markers in DM rats with autologous transplantation of adipose-derived stem cells (ADSC) with and without epigallocatechin gallate (EGCG) precondition;

FIG. 15 shows the Western blot analysis for protein expression amount of cardiac apoptosis protein markers in DM rats with autologous transplantation of adipose-derived stem cells (ADSC) with and without epigallocatechin gallate (EGCG) precondition;

FIG. 16 shows the investigation of cardiac apoptosis protein markers (green spots) in DM rats with autologous transplantation of adipose-derived stem cells (ADSC) with and without epigallocatechin gallate (EGCG) precondition;

FIG. 17 shows the investigation of cardiac longevity protein markers in DM rats with autologous transplantation of adipose-derived stem cells (ADSC) with and without epigallocatechin gallate (EGCG) precondition;

FIG. 18 illustrates the Western blot analysis for protein expression amount of cardiac echo and hypertrophy protein markers in DM rats with autologous transplantation of adipose-derived stem cells (ADSC) with and without epigallocatechin gallate (EGCG) precondition, (A) observation of interventricular septal thickness at end diastole (IVSd), (B) observation of interventricular septal thickness at end systole (IVSs);

FIG. 19 illustrates the Western blot analysis for protein expression amount of cardiac echo and hypertrophy protein markers in DM rats with autologous transplantation of adipose-derived stem cells (ADSC) with and without epigallocatechin gallate (EGCG) precondition, (A) observation of left ventricular posterior wall thickness at end diastole, (LVPWd), (B) observation of left ventricular posterior wall thickness at end systole (LVPWs);

FIG. 20 illustrates the Western blot analysis for expression amount of echocardiography analysis related proteins and hypertrophy protein markers in DM rats after autologous transplantation of adipose-derived stem cells (ADSC) with and without epigallocatechin gallate (EGCG) precondition;

FIG. 21 shows the investigation of Masson Trichrome tissue slices of cardiac tissue in each groups to analyze the cardiac fibrosis protein markers in DM rats with autologous transplantation of adipose-derived stem cells (ADSC) with and without epigallocatechin gallate (EGCG) precondition;

FIG. 22 shows Western blot analysis of protein expression amount of cardiac fibrosis related protein markers to analyze the fibrosis related proteins in homogeneous cardiac tissue in DM rats with autologous transplantation of adipose-derived stem cells (ADSC) with and without epigallocatechin gallate (EGCG) precondition;

FIG. 23 shows the summary of the test result.

### DETAILED DESCRIPTION OF THE INVENTION

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

The present invention provides a therapeutic compound for repairing cardiac injuries, wherein the therapeutic compound comprises a stem cell and the epigallocatechin gallate (EGCG) of green tea are pretreated, so as to increase the differentiation ability of the stem cell.

The following embodiments illustrate the present invention but not limited the scope of the present invention.

### EXAMPLE 1

### The Preparation of the Rat Adipose-Derived Stem Cells and the Experiments Thereof

The adipose-derived stem cells were taken out from the abdominal adipose tissue of 8-month-old Wistar male rats via surgery. After cutting the adipose tissues into suitable sizes, the adipose tissues were washed by antibiotic containing saline. The washed adipose tissues were placed into a Type II collagenase (0.01%) containing saline, and were stirred and heated under 37° C. water bath for 1 hour. Then the adipose tissues were centrifuged by 3000 rpm under room temperature for 10 minute, the precipitants were extracted for cell culture in the culture dish.

1. Identification of Adipose-Derived Stem Cells

The adipose-derived stem cells were cultured to passage 2 then transfused into the rat via the tail vein for autologous transplantation. Before the autologous transplantation, the cultured adipose-derived stem cells were identified to confirm the transplants cells were stem cells. There were two kinds of methods used for stem cells identification in this experiment, one is identifying the positive marks and negative marks on the adipose-derived stem cells membrane, wherein the positive makers would be existed definitely on the stem cells; in contrast, the negative markers would definitely not be existed on the stem cells.

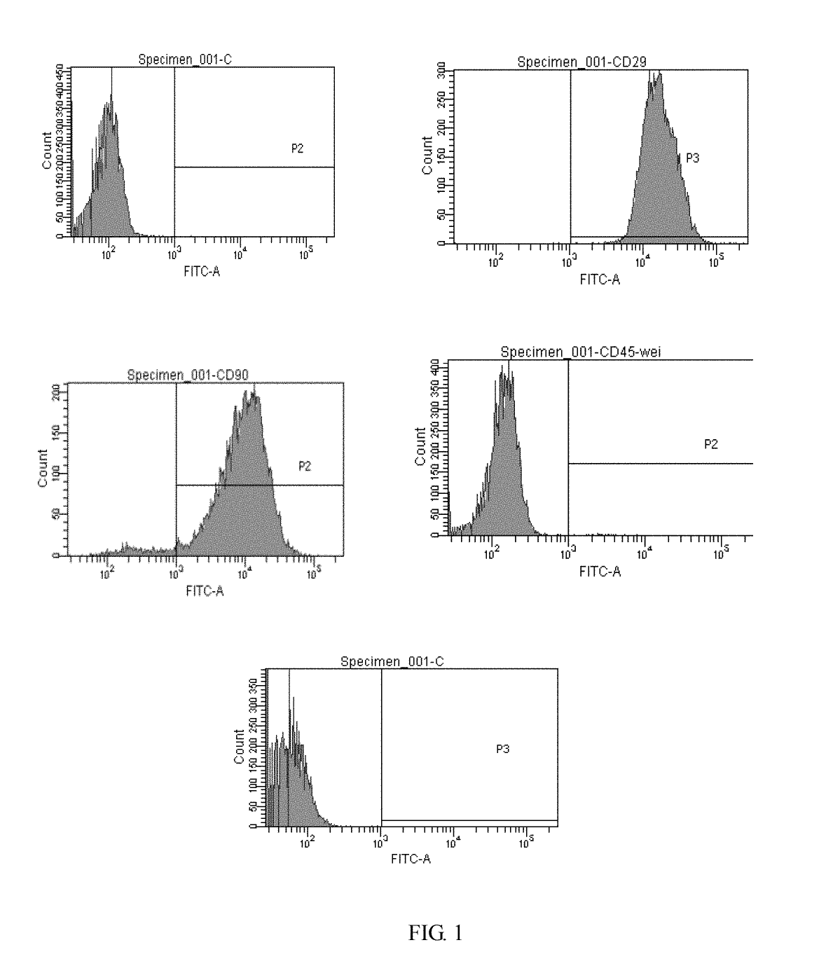

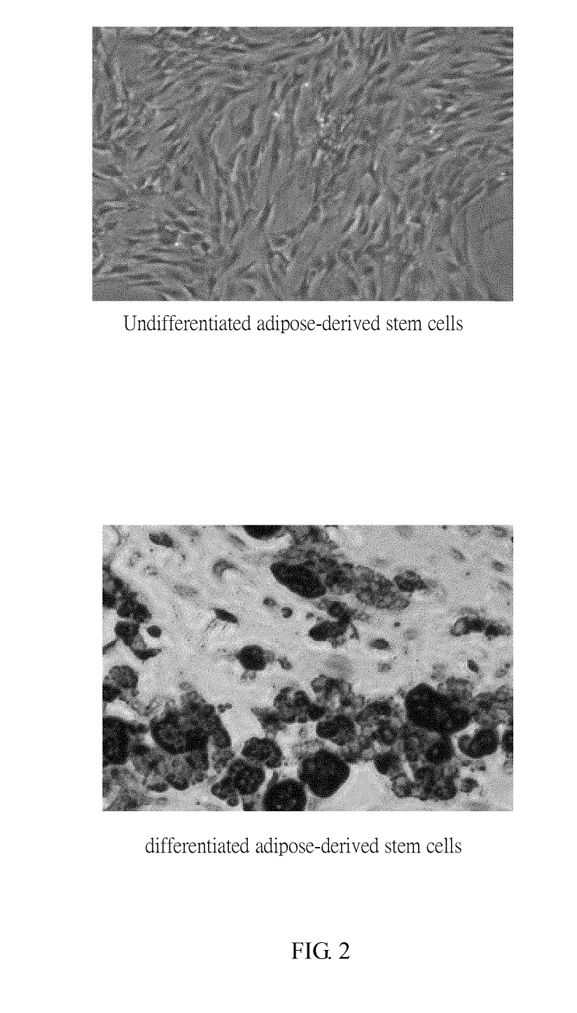

As shown in FIG. 1, the expression amount of positive markers CD90 and CD29 on the stem cells membrane was 95% and 98% respectively; on the other hand, the expression amount of negative markers CD45 and CD31 was 0.5% and 0.5% on the stem cells membrane respectively. In addition to identification of positive and negative marks on the stem cells membrane, the stem cells must be proven to have the ability of differencing into other cells types. As shown in FIG. 2, in the differentiation test result showed that the stem cells have the ability to differentiate into the adipose-derived stem cells (ADSC).

2. Gene and siRNA Transfection

The stem cells were cultured in DMEM medium until the stem cells grow up to 80% full, then the siRNA, target plasmid and DharmaFECT Duo transfection reagent (Dharmacon, Inc.) were added for transfection experiment. The 3.5 L plasmid (2 g/L) and 35 L siRNA (20M) were mixed in 7001 serum-free DMEM medium (A tube); while the DharmaFECT Duo reagent were mixed with serum-free DMEM culture medium by ratio of 1:50 for 5 minutes (B tube). Then A tube and B tube were mixed and placed for 20 minutes. The same amount of above mentioned tube A and tube B mixture was added into a cells containing petri dish, and the mixture was transfected at 37° C. in an incubator, and then the cells were collected to have further analysis.

3. Protein Assay

Bradford protein assay method was used to quantify the protein amount in this experiment, wherein the principle of this method is that the protein would form a blue complex with Coomassie brilliant blue G-25, while the darker blue color represents higher protein content. First, one-fifth volume of Bradford protein dye was added into a series of known concentration of BSA, then the absorbance of visible light of a wavelength at 595 nm was measured to obtain a standard curve, then the O.D. values of the samples were measured in the same way, and the protein concentrations were obtained according to the standard curve.

4. The Western Blot

After the drug treatment, the culture medium was removed from the cells and rinsed with PBS buffer for three times; then 1 mL PBS was used to scrape the cells from the dish and the cells containing solution was placed in a centrifuge tube, and the solution was centrifuged by 12,000 rpm for 10 minutes, then the supernatant was removed, and the lysis buffer (50 mM Tris pH 7.5,0.5 M NaCl, 1.0 mM EDTA pH 7.5,1 mM BME, 1% NP40,10% glycerol, protease inhibitor cocktail table) was added and mixed. The mixture was placed on ice and shock once every 5 minutes for 30 minutes, and then the mixture was centrifuged at 12,000 g in 4° C. for 10 minutes, the upper layer was placed in a new tube to measure the protein concentration.

Extraction of Cytoplasmic Cytochrome c

After the drug treatment, the culture medium was removed from the cells and rinsed with PBS buffer for three times; then 1 mL PBS was used to scrape the cells from the dish and the cells containing solution was placed in a centrifuge tube, and the solution was centrifuged by 12,000 rpm for 10 minutes then the supernatant was removed and extraction buffer (50 mM Tris pH 7.5-0.5 M NaCl-1.0 mM EDTA pH 7.5-10% glycerol-protease inhibitor cocktail table) was added into the mill tube, then grinded on ice. And the homogenate was then placed in a new centrifuge tube to centrifuge at 4° C. by 12,000 rpm for 10 minutes, the upper layer was placed in a new centrifuge tube for measuring the protein concentration.

40 g protein samples were added into a PBS solution, and 5× loading dye was added then mixed evenly and boiled for 10 minutes, then analyze by SDS-polyacrylamide slab gel electrophoresis. The upper layer of the SDS-polyacrylamide gel electrophoresis was 3.75% Stacking gel, and the lower layer was 5% and 12% Separating gel. The plastic plates was fixed to the electrophoresis apparatus and the electrophoresis buffer was filled into the electrophoresis tank, and then the treated protein sample was added into the U-shaped groove formed on a plastic plate, and undergo electrophoresis with 75 volts. The protein was transfer after the termination of electrophoresis, the gel colloidal was tiled on a moistened Whatman 3M filter paper, in the mean time, the previously methanol-soaked PVDF membrane was used to cover on the above colloid, and then covered with a wet 3M filter paper, and then glass rod was used to catch the bubbles and loaded into a transfer Holder, then placed in a electrotransfer Tank (containing a transfer buffer) at 4° C., transferred 1 hour with 100 volts transfer power. Then, PVDF membrane was removed and immersed with Blocking buffer (contains 5% (w/v) skim milk (PBS-non-fat milk powder)) was shaken at room temperature for one hour. The PVDF membrane reacted with primary antibody at 4° C. refrigerator overnight, then washed twice with washing buffer, each time for 10 minutes, and finally washed once and discarded. Then the sample was reacted with Horseradish peroxidase conjugated secondary antibody for 2 hours, and then washing the PVDF membrane in the same way. Finally, the PVDF membrane was immersed in 4 mL substrate solution (substrate buffer) for color reaction.

5. Cell Viability Analysis

The cells were cultured in a 24-well culture dish, after the cells were treated with drugs, the culture medium was removed and rinsed with PBS buffer for 3 times. The culture medium was replaced in 0.5 mg/ml MTT containing culture medium, and cultured for about 3 to 4 hours, then the culture medium was removed and rinsed with PBS buffer, and 1 mL isopropanol was added to dissolve purple formazan crystalline, the O.D. 570 nm absorbance was measured after 5 minutes.

6. DAPI (4,6-diamidino-2-phenylindole) Staining Fluorescent Cells

After the drug treatment, the medium was removed and rinsed with PBS buffer (3 times), and then the cells were fixed in 4% paraformaldehyde at room temperature for 30 minutes, washed three times with PBS buffer to remove the paraformaldehyde. DAPI (4,6-diamidino-2-phenylindole) (1 μg/mL) was used for cell staining for 30 minutes and then washed with PBS for three times, a fluorescence microscope was used to observe the 340/380 nm excitation wavelength with 100× photographic archives.

7. Analysis of Cell Apoptosis

After the drug treatment, the medium was removed and rinsed with PBS buffer (3 times), and then the cells were fixed in 4% paraformaldehyde at room temperature for 30 minutes, washed three times with PBS buffer to remove the paraformaldehyde. The permeabilisation solution (0.1% Triton X-100 in 0.1% sodium citrate) was added and reacted for 2 minutes at 4° C., and then washed with PBS for three times. The cells were treated with TUNEL reaction mixture (label solution+enzyme solution) for 1 hour, fluorescence microscope was used to observe 450-500 nm excitation wavelength observation cells with 100× photographic archive.

8. Green Tea EGCG Strengthen Adipose Stem Cells the Ability to Experiment

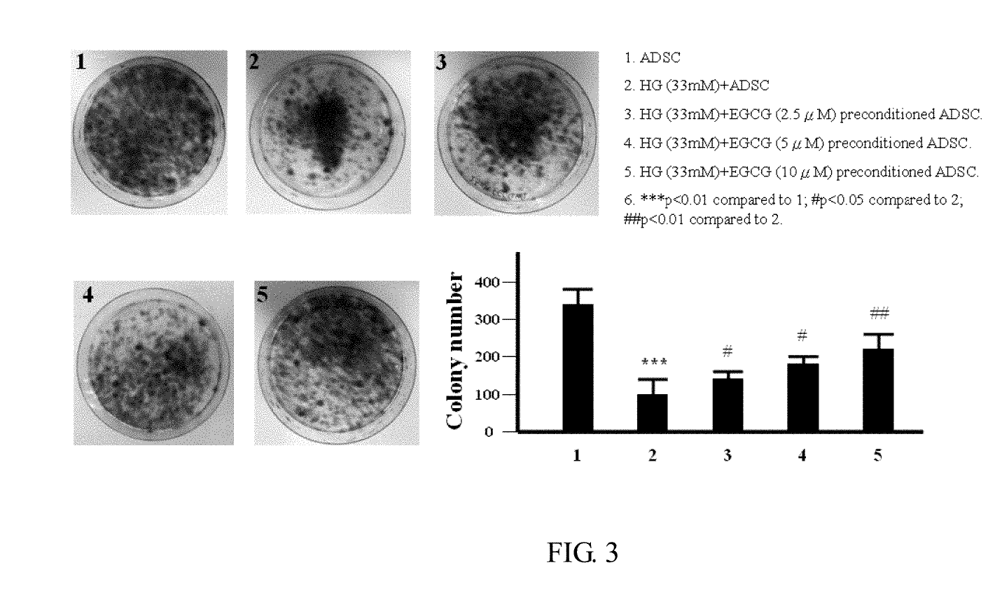

In the aspect of stem cell proliferation experiment, the more the number of stem cell colonies in different experimental conditions represents for the better growth experiment condition for stem cells. The stem cells were divided into 5 different groups, which were: Group 1: stem cell group, Group 2: high glucose destroyed stem cell group, Group 3: EGCG (2.5 μM in concentration) precondition high glucose destroyed stem cell group, Group 4: EGCG (5 μM in concentration) precondition high glucose destroyed stem cell group, Group 5: EGCG (10 μM in concentration) precondition high glucose destroyed stem cell group. As shown in FIG. 3, the colonies of the five groups were 338±38, 100±26,152±17,178±22 and 226±31 respectively. In comparison with the normal stem cell group (Group 1), the distribution of the stem cells colonies cultured in high glucose medium was inhibited (Group 1>Group 2, p<0.001). In contrast, in comparison to the high glucose destroyed stem cells, the stem cell colonies were recovered under different EGCG concentration precondition (Group 2<Group 3, p<0.05; Group 2<Group 4, p<0.05; Group 2<Group 5, p<0.01). Then the migration ability of stem cells under different experiment conditions was tested. The more numbers of stem cells represent the stem cells have stronger migration ability under this experiment condition.

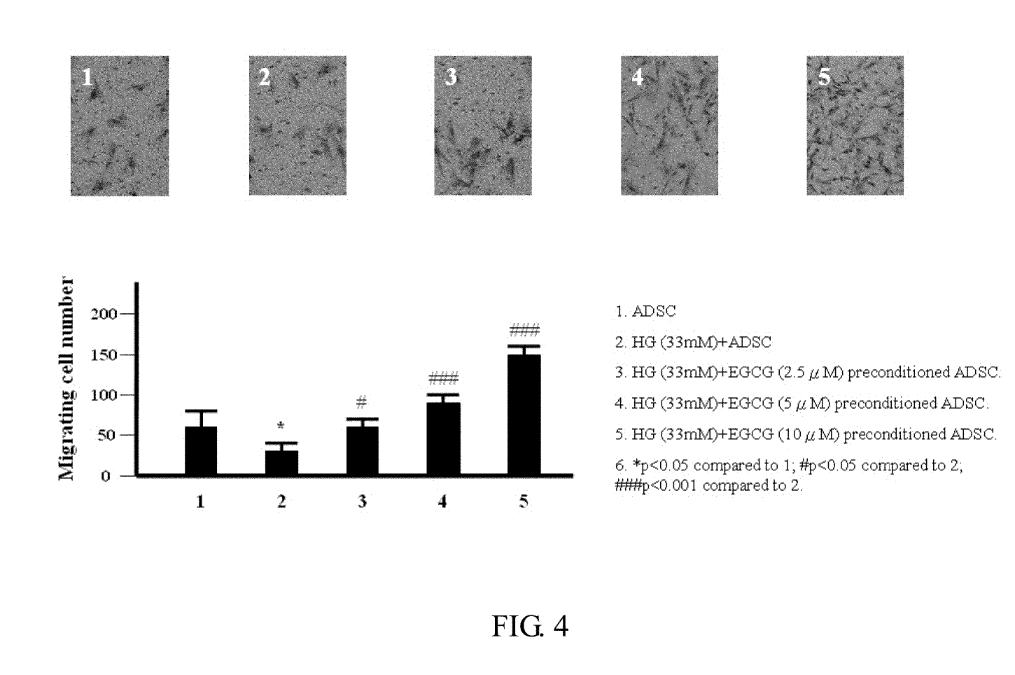

The stem cells were divided into five different groups, which were: Group 1: stem cell group, Group 2: high glucose destroyed stem cell group, Group 3: EGCG (2.5 μM in concentration) precondition high glucose destroyed stem cell group, Group 4: EGCG (5 μM in concentration) precondition high glucose destroyed stem cell group, Group 5: EGCG (10 μM in concentration) precondition high glucose destroyed stem cell group. FIG. 4 showed the test result of stem cell migration ability. The migration ability of the five groups was: 63±10, 36±7, 55±5, 84±6 and 144±4, respectively. In comparison with the normal stem cell group (Group 1), the migration ability of the stem cells cultured in high glucose medium was decreased (Group 1>Group 2, p<0.05). In contrast, in comparison to the high glucose destroyed stem cells, the migration ability of stem cells was recovered under different EGCG concentration precondition (Group 2<Group 3, p<0.05; Group 2<Group 4, p<0.001; Group 2<Group 5, p<0.001). Then the migration ability of stem cells under different experiment conditions was tested. The more numbers of stem cells represents the stem cells have stronger migration ability under this experiment condition.

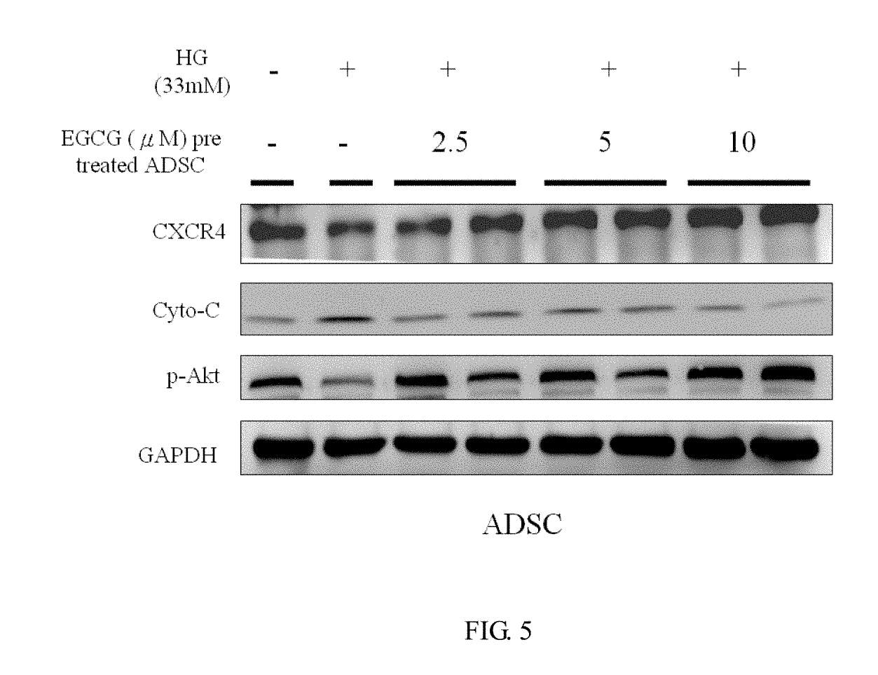

FIG. 5 showed the analysis of protein expression of stem cells under different experiment conditions. In comparison with the normal stem cells group (Group 1), the migration protein (CXCR4) expression level of stem cells cultured in high glucose medium was decreased. In contrast, in comparison to the high glucose destroyed stem cells, the migration protein (CXCR4) expression level of stem cells was recovered under different EGCG concentration precondition. The expression level of survival related protein p-Akt was similar to CXCR4. In comparison with the normal stem cells group (Group 1), the apoptosis protein cytochrome-C expression level of stem cells cultured in high glucose medium was increased. In contrast, in comparison to the high glucose destroyed stem cells, the apoptosis protein cytochrome-C expression level of stem cells was decreased under different EGCG concentration precondition.

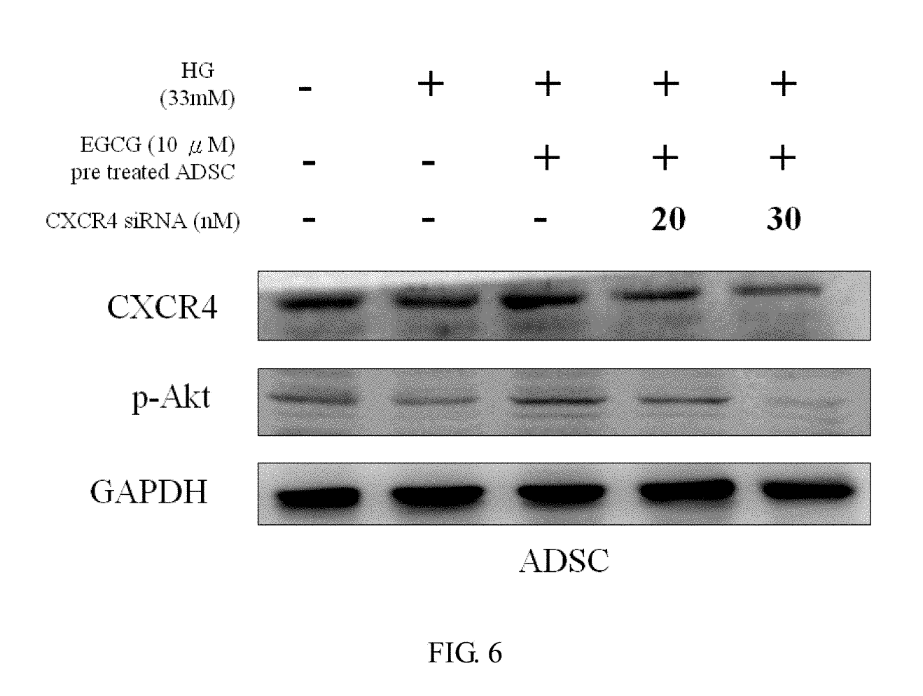

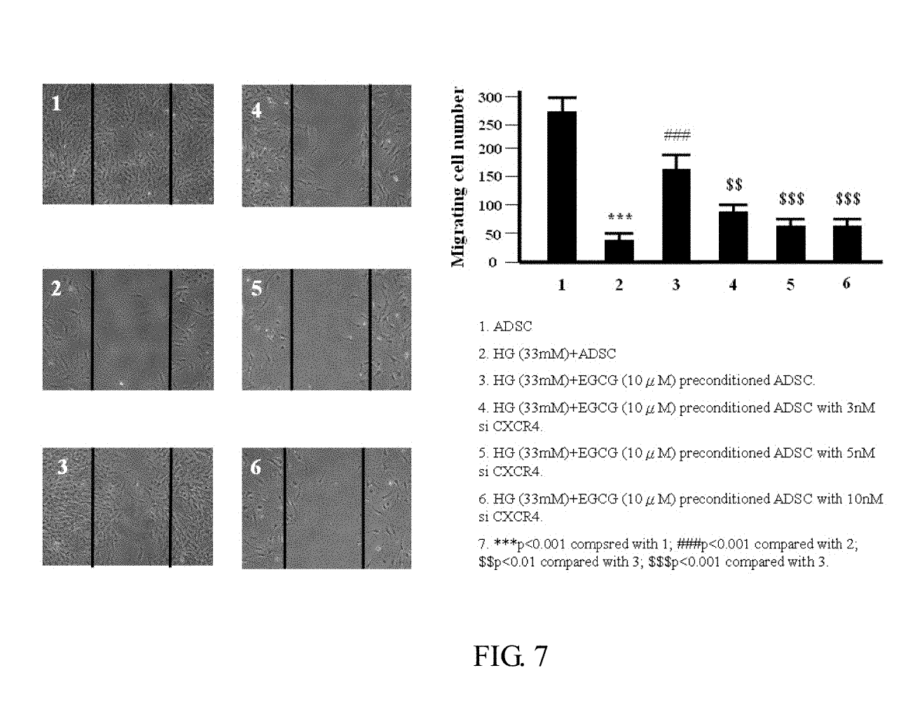

FIG. 6 showed the protein expression of stem cells under different experiment conditions. During the analysis of protein expression level, the EGCG induced expression level of CXCR4 and p-Akt increase would be eliminated by siRNA CXCR4. FIG. 7 showed the migration ability of stem cells, so as to investigate the numbers of migrated stem cells under different experiment conditions. The stem cells were divided into six groups in this test, which were: Group 1: stem cell group, Group 2: high glucose destroyed stem cell group, Group 3: EGCG (10 μM in concentration) precondition high glucose destroyed stem cell group, Group 4: siRNA CXCR4 (3 nM) was added in Group 3, Group 5: CXCR4 siRNA (10 nM) was added into Group 4. The numbers of migrated stem cells were 288±25, 36±7, 159±17, 84±6, 41±8 and 40±2 respectively. Wherein the number of migrated stem cells in Group 2 was significantly less than Group 1 (p<0.001), the number of migrated stem cells in Group 3 was significantly more than Group 2 (p<0.001), the number of migrated stem cells in Group 4 was significantly less than Group 3 (p<0.01), the number of migrated stem cells in Group 5 was significantly less than Group 3 (p<0.001), the number of migrated stem cells in Group 6 was significantly less than Group 3 (p<0.001).

The following experiments were investigating the regeneration function of stem cells when the H9c2 cardiomyocytes was under destroy of high glucose.

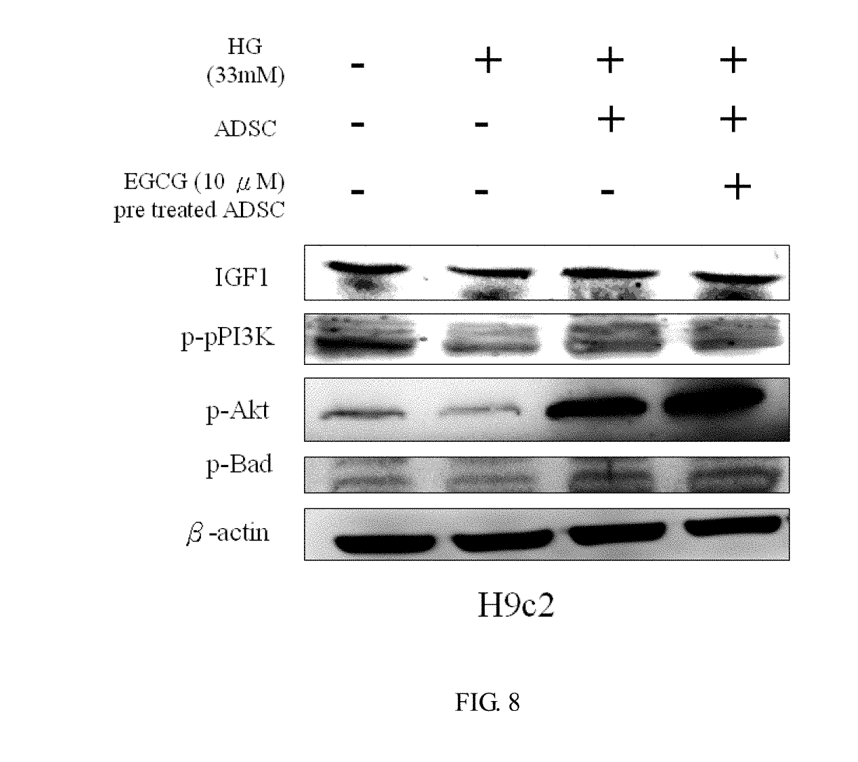

FIG. 8 showed the survival related protein expression level of H9c2 cardiomyocytes under different experiment conditions. In comparison with the normal cell group (row 1), the expression level of cell survival related protein, such as IGF1, PI3K, Akt and p-Bad was decreased under destroy of high glucose (row 2). The expression level of these cell survival related proteins was recovered while the stem cells were added (row 3), and expression level of these cell survival related proteins in EGCG precondition stem cells added group (row 4) was higher than the untreated stem cells (row 1). FIG. 2G showed the expression level of cell survival protein p-Akt in co-cultured H9c2 cardiomyocytes and stem cells under different experiment conditions. The high glucose could reduce the expression level of cell survival protein p-Akt, and the expression level of cell survival protein p-Akt was increased in the stem cells added group or the EGCG precondition group; however, after CXCR4 siRNA was added, the regeneration effect of stem cells and EGCG precondition stem cells would be inhibited.

### EXAMPLE 2

### 0056

### Animal Experimental Design and Analysis

2-month-old Wistar male rats (purchased from Green Seasons Company) were divided into four groups, which were: normal control group, the STZ (55 mg/kg) induced diabetes group, diabetes with autologous adipose stem cells treatment group, and diabetes with EGCG green tea precondition and autologous adipose stem cells treatment group. The experiment results were shown as below figures. Rats were kept in animal cages under the cycles of 12 hours daytime and 12 hours night-time. The eating and drinking during the feeding was freely up to the rats in the animal cage. Two rats were raised in one animal cage, and animal sook materials were changed every two days during the feeding period. When the blood glucose of rats in the diabetes group increased up to 200 mg/dl, the rats would be identified as having diabetes symptoms, and the rats in this group would be treated by autologous stem cell transplantation therapy after one month. The autologous stem cells transplantation was through administrating 1×10⁵ stem cells via tail vein.

1. Analysis of Animal Serum and Body Weight

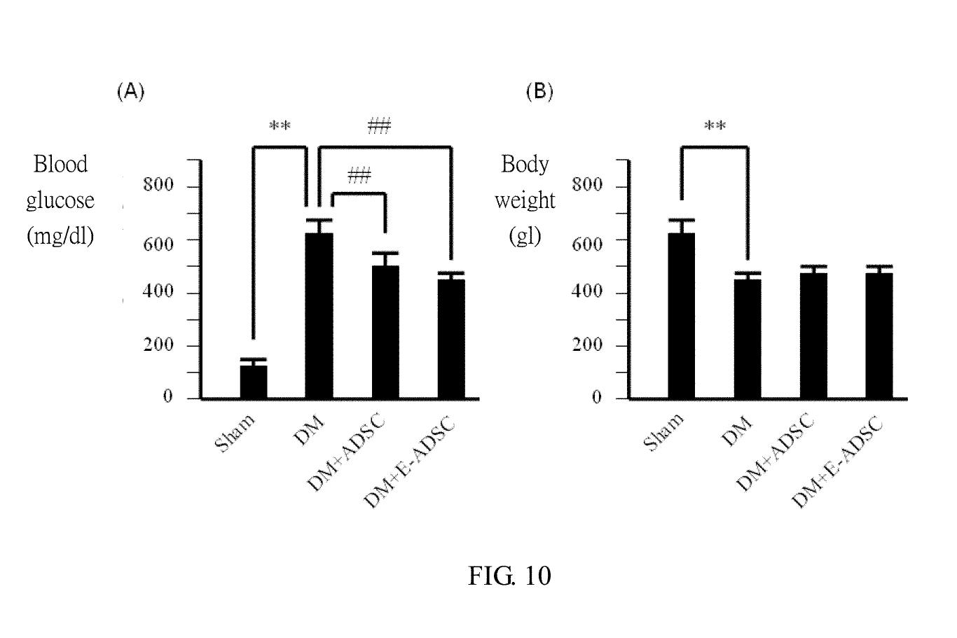

The rats were divided into four groups, which were: normal group (sham), diabetes mellitus group (DM), adipose-derivate stem cells therapy in diabetes mellitus group (DM+ADSC), and EGCG pretreated stem cells to treat diabetes mellitus (DM+E-ADSC). FIG. 10A showed the serum of the animal was analyzed for the blood glucose level after sacrificing the animals in the end of experiments. The blood glucose level of sham group, DM group, DM+ADSC group and DM+E-ADSC group were 126±9 mg/dl, 611±35 mg/dl (compared with sham group, p<0.01), 493±37 mg/dl (compared with DM group, p<0.01) and 451±16 mg/dl (compared with DM group, p<0.01). The weights of the rats in sham group, DM group, DM+ADSC group and DM+E-ADSC group were 627±46 g, 438±28 g (compared with sham group, p<0.05), 473±6 g and 477±13 g (shown in FIG. 10B) respectively.

2. Animal Echocardiography Analysis

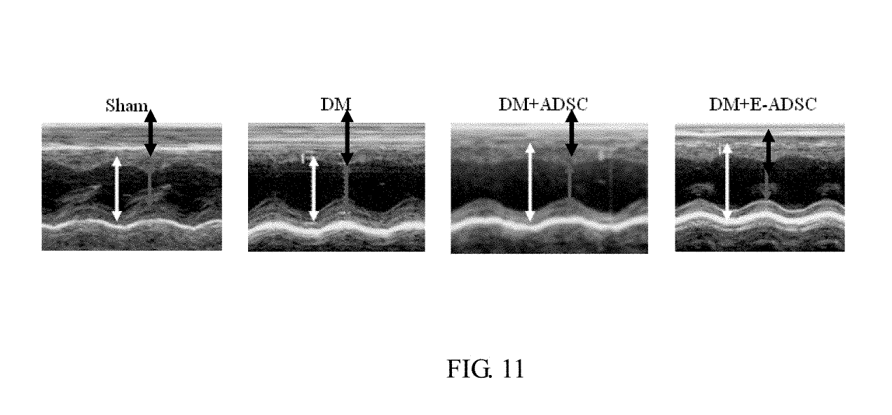

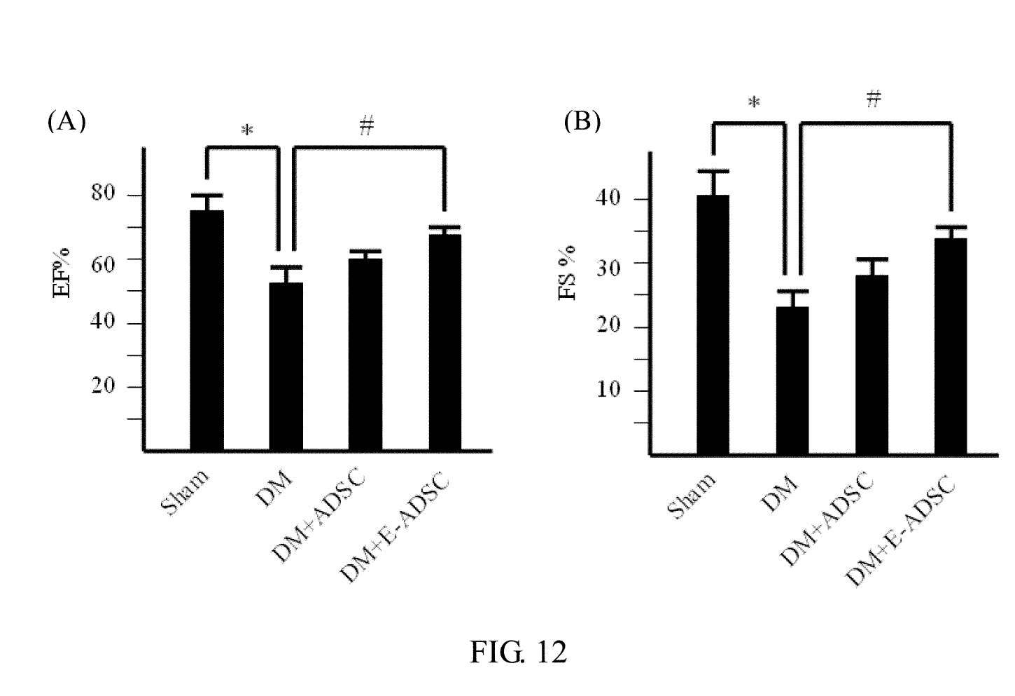

Animal echocardiography analysis was commissioned according to standard operating procedures by the cardiologist in China Medical University Hospital. FIG. 11˜FIG. 12 showed the analysis result of rat echocardiography, which represented the analysis result of rats heart function in different groups. FIG. 11 showed the analysis result of rat echocardiography, wherein the red arrow red arrow indicates the heart systolic power, the longer the red arrow indicate spoor heart systolic power. In comparison with sham group, red arrows in the DM group were longer, which represented the heart systolic power of rats in DM group was poor than the heart systolic power of rats in sham group; the red arrows in the DM+ADSC group and DM+E-ADSC group were shorter, which represented the heart systolic power in rats of DM+ADSC group and DM+E-ADSC group were stronger than the heart systolic power of rats in DM group. FIG. 12 showed the test result of cardiac blood ejection fraction (EF %), wherein the higher EF % represented better heart function. The cardiac blood ejection fraction (EF %) of sham group, DM group, DM+ADSC group and DM+E-ADSC group were 75±4%, 52±5% (compared with the sham group, p<0.05), 60±3% and 68±1% (compared with DM group, p<0.05). FIG. 12 illustrated the test result of fractional shortening (FS %), the higher FS % represented better heart function. The fractional shortening (FS %) of sham group, DM group, DM+ADSC group and DM+E-ADSC group were 41±3%, 24±3% (compared with the sham group, p<0.05), 28±2% and 34±1% (compared with DM group, p<0.05).

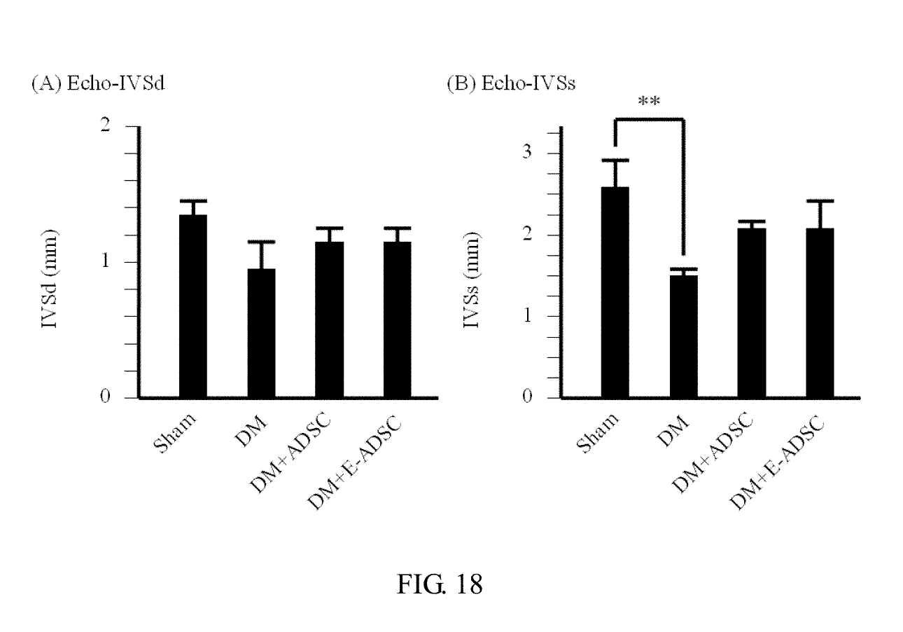

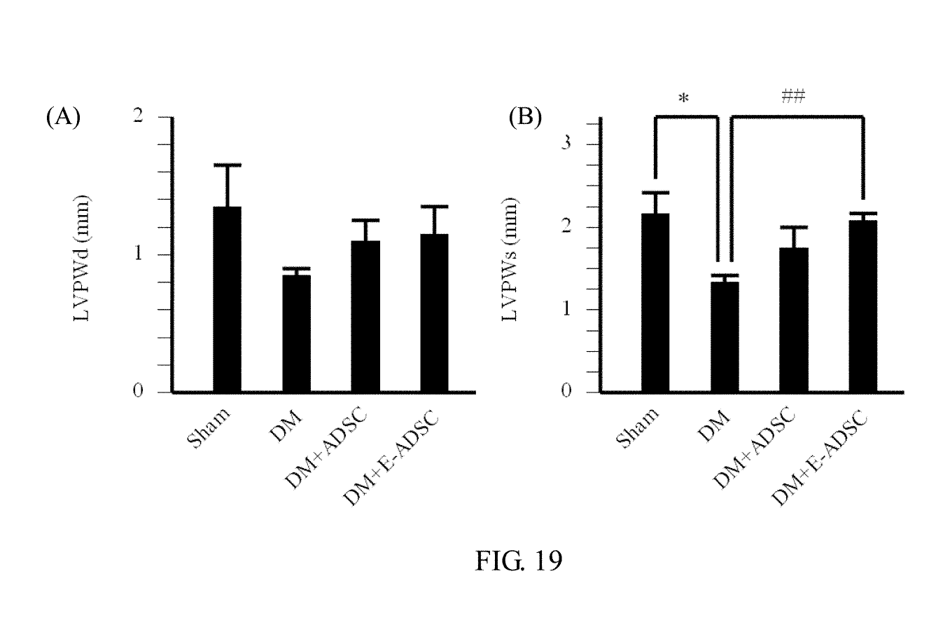

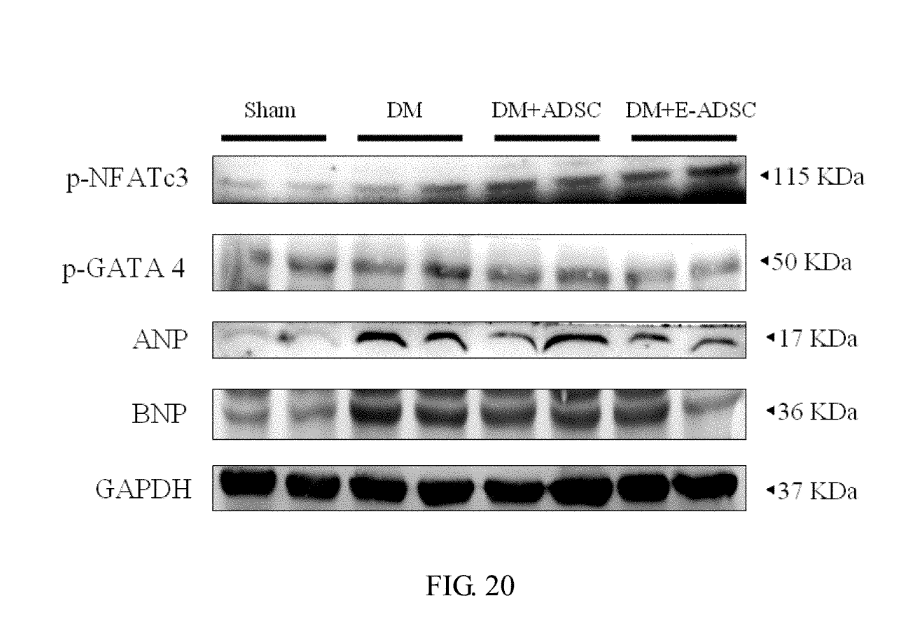

To investigate the pathway of animal heart tissue hypertrophy, the change of left ventricle represented functional change of heart. The rats were investigated by echocardiography before sacrifice, so as to analyze the effect of diabetes on left ventricle and the effect of stem cells on left ventricle regeneration. FIG. 18˜20 showed the test result of rats echocardiography on left ventricle. FIG. 18A showed the interventricular septal thickness at end diastole (IVSd), the values of each groups were sham=1.36±0.1 mm, DM=0.98±0.2 mm, DM+ADSC=1.22±0.1 mm and DM+E-ADSC=1.2±0.1 mm. FIG. 18B showed the interventricular septal thickness at end systole (IVSs), values of each groups were sham=2.68±0.2 mm, DM=1.49±0.1 mm (sham group and p<0.01), DM+ADSC=2.14±0.1 mm (DM group and p<0.05) and DM+E-ADSC=2.26±0.3 mm (DM group and p<0.05). FIG. 19 showed the investigation result of Left ventricular posterior wall thickness at end diastole (LVPWd), the values of each group were sham=1.36±0.4 mm, DM=0.85±0.1 mm, DM+ADSC=1.11±0.2 mm and DM+E-ADSC=1.12±0.3 mm. FIG. 19 showed the investigation results of Left ventricular posterior wall thickness at end systole (LVPWs), the values of each groups were sham=2.19±0.2 mm respectively, DM=1.3±0.1 mm (sham group and p<0.05), DM+ADSC=1.5±0.3 mm and DM+E-ADSC=2.18±0.1 mm (DM group and p<0.001). FIG. 20 showed the expression level analysis results of myocardial hypertrophy associated proteins, compared with the sham group, the expression level of hypertrophy associated proteins such as p-GATA4, ANP and BNP in DM group was significantly increased; expression level of hypertrophy associated proteins in DM+ADSC group and DM+E-ADSC group was significantly much lower than the DM group; the expression level of hypertrophy associated proteins in DM+E-ADSC group was the lowest. In contrast, the expression level of non hypertrophy associated protein p-NFATc3 showed inversely trend with the expression level of hypertrophy associated proteins.

3. The Heart Tissue Slices, Staining and Analysis

Heart tissue slices, staining and analysis was commissioned according to standard operating procedures by the Department of Pathology in Changhua Christian Hospital. The heart tissue were sliced and stained after sacrificing the rats in the end of experiments, so as to investigate the arrangement of cardiomyocytes and the gap size between cardiac tissues. The arrangement of cardiomyocytes would be disorder and the gap between cardiac tissues would become larger when the heart was injured.

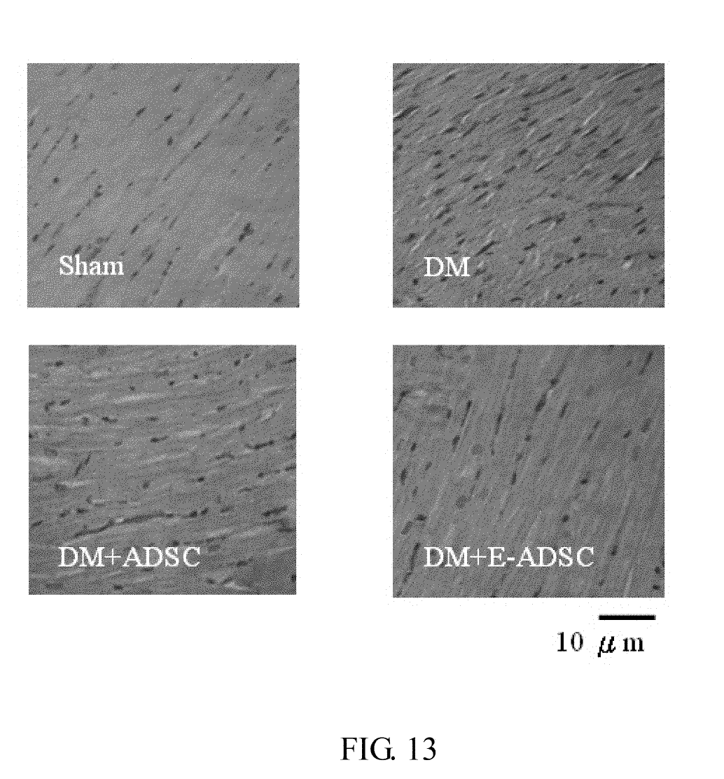

FIG. 13 showed the HE staining analysis of animal cardiac tissue slices. In comparison with sham group, the HE staining of animal cardiac tissue slices in DM group showed the arrangement of cardiomyocytes (blue dots) was disorder and the gap size (white space) between cardiac tissues was larger; in comparison with DM group, the HE staining of animal cardiac tissue slices in treatment group (DM+ADSC group and DM+E-ADSC group) showed arrangement of cardiomyocytes (blue dots) was regular and the gap size (white space) between cardiac tissues was smaller.

### EXAMPLE 3

### Cardiac Cardiomyocytes Cell Culture and Analysis

The embryonic rat cardiocardiomyocytes transition cell lines H9c2 cells (from ATCC CRL-1446) and adipose-derived stem cells were cultured in a 10% fetal bovine serum (FBS, Hyclone), 1% Antibiotic-Antimycotic (Gibco) containing Dulbeco's Modified Eagle Medium (DMEM, Sigma), the incubator was set at 5% CO2, 37° C. The culture medium was changed 2-3 times every week. The serum-free medium was used for culturing cardiomyocytes overnight, then cardiomyocytes were treated with drugs in different time schedule or drug concentrations.

1. The Cell Survival Related Proteins Analysis of Animal Cardiomyocytes

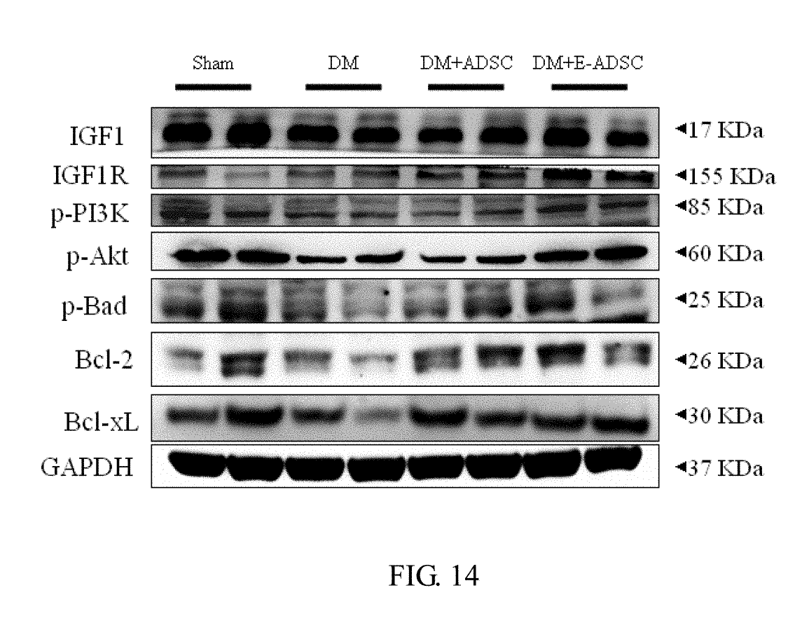

The rats hearts were isolated and homogenized in the end of the experiment, and then analyze the expression level of cell survival related proteins in rat cardiac tissues by Western blot. As shown in FIG. 14, in comparison with sham group, the expression level of cell survival related proteins was significantly decreased in DM group; in comparison with DM group, the expression level of cell survival related proteins was significantly higher in treatment group (DM+ADSC group and DM+E-ADSC group); in further observation, the expression level of cell survival proteins such as IGF1R, p-PI3K and p-Akt, in DM+E-ADSC group was even higher than DM+ADSC group.

2. Analysis of Animal Myocardial Cell Apoptosis Proteins

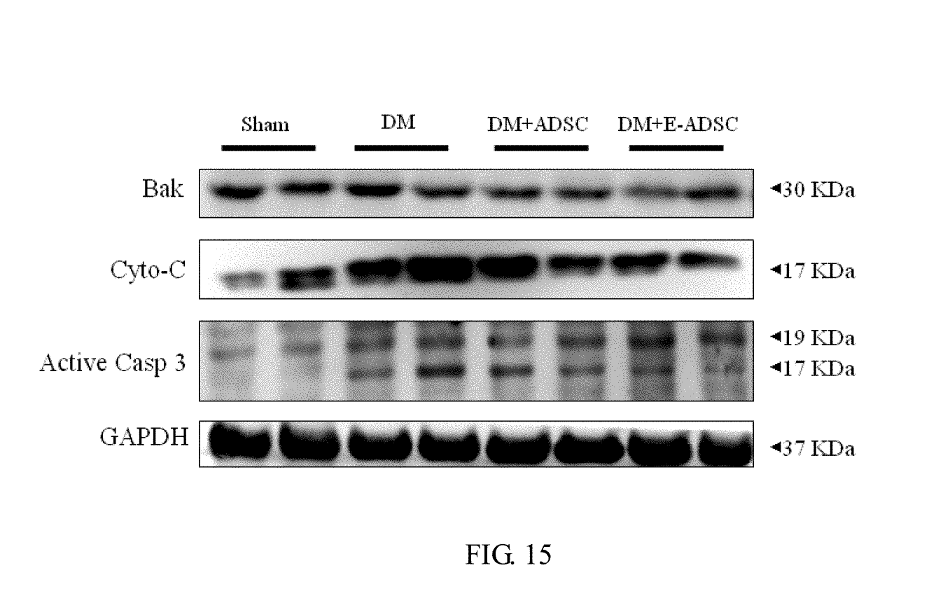

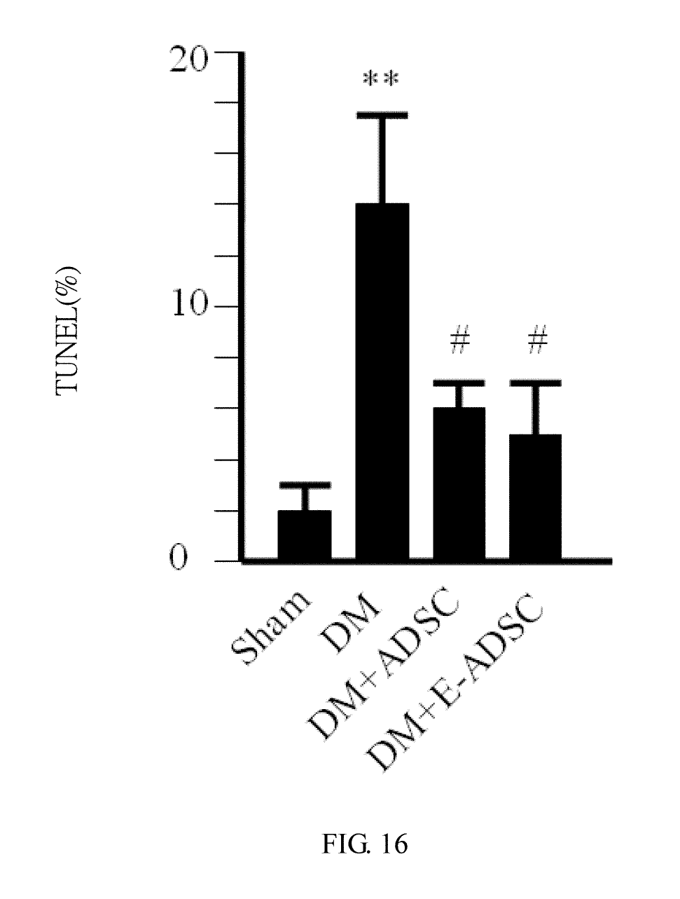

The rats hearts were isolated and homogenized in the end of the experiment, and then analyze the expression level of cell apoptosis related proteins in rat cardiac tissues by Western blot. As shown in FIG. 15, in comparison with sham group, the expression level of cell apoptosis related protein was significantly increased in DM group; in comparison with DM group, the expression level of cell apoptosis related protein was significantly lower in treatment group (DM+ADSC group and DM+E-ADSC group); in further observation, the expression level of cell apoptosis proteins in DM+E-ADSC group was even lower than DM+ADSC group. FIG. 16 showed the observation results of TUNEL staining of cardiomyocytes to investigate the cell apoptosis. The TUNEL staining method was using two dyes TUNEL and DAPI to stain cardiomyocytes, wherein the DAPI (blue dots) was used for cardiomyocyte nucleus staining for analyze the numbers of cardiomyocytes; the TUNEL (green dots) was used for apoptotic cardiomyocytes, thus the green fluorescence on the apoptotic cardiomyocytes would be detected. As shown in FIG. 16, the numbers of apoptotic signals (green dots) in sham group, DM group, DM+ADSC group and DM+E-ADSC group were 2±1%, 14±4%, 6±1% and 5±2%. In comparison with sham group, the green fluorescence signals were significantly more in DM group (sham<DM, p<0.01), and the green fluorescence signals were significantly less in treatment group (DM+ADSC group and DM+E-ADSC group; DM>DM+ADSC, p<0.05; DM>DM+E-ADSC, p<0.05).

3. Animal Cardiomyocytes Sirt1 Associated Protein Analysis

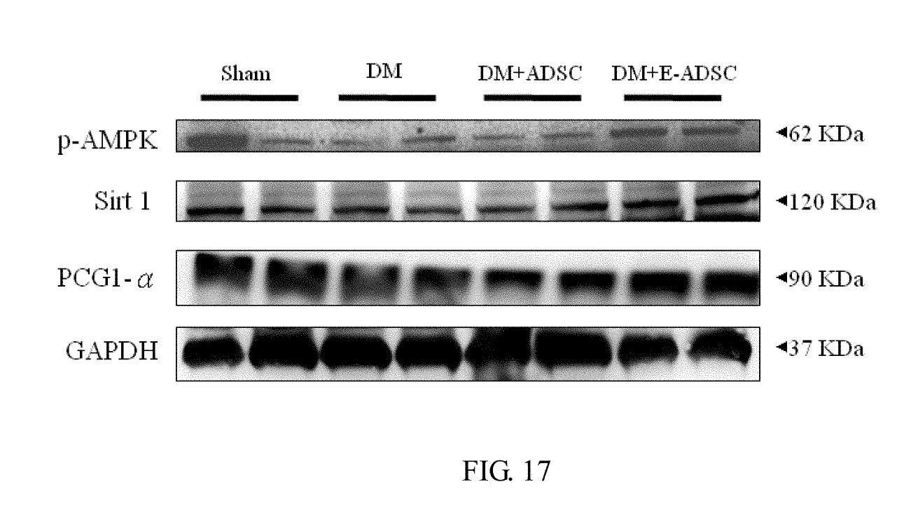

The rats hearts were isolated and homogenized in the end of the experiment, and then analyze the expression level of Sirt1 related proteins in rat cardiac tissues by Western blot. As shown in FIG. 17, in comparison with sham group, the expression level of Sirt1 related protein was significantly decreased in DM group; in comparison with DM group, the expression level of Sirt1 related protein was significantly higher in treatment group (DM+ADSC group and DM+E-ADSC group); in further observation, the expression level of Sirt1 related proteins in DM+E-ADSC group was even higher than DM+ADSC group.

4. Investigation of the Animal Heart Tissue Fibrosis Pathway

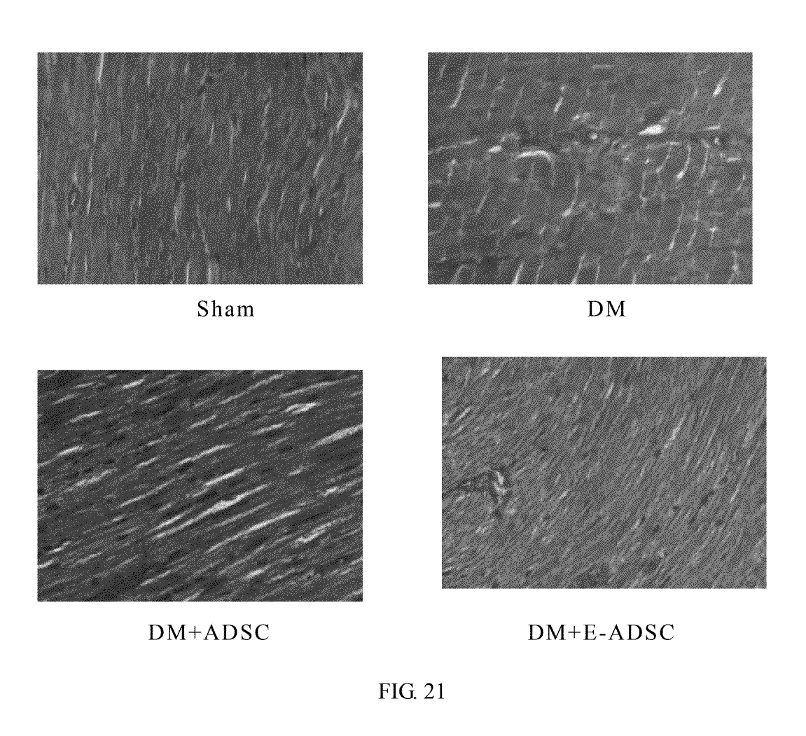

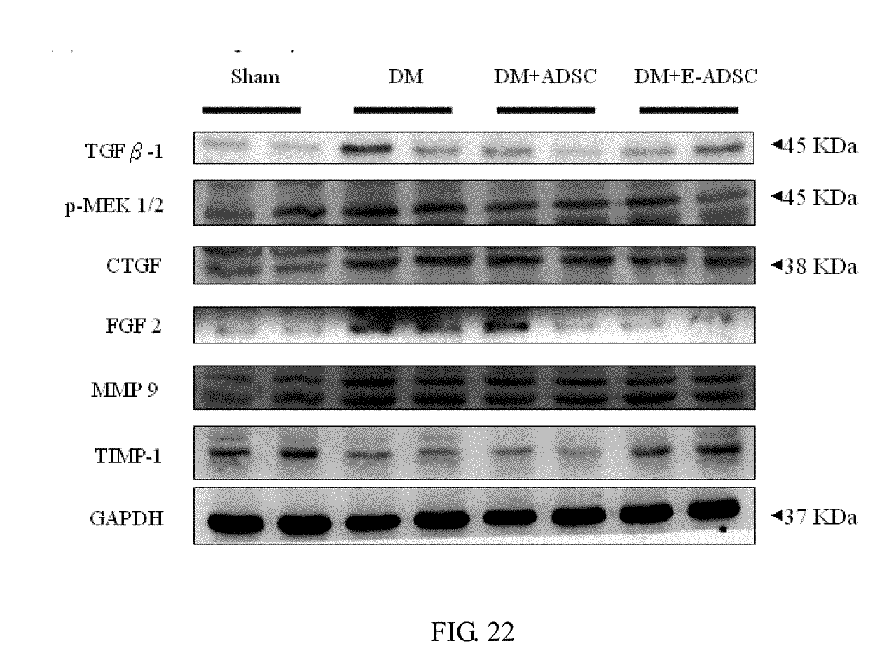

The heart tissues were sliced and Masson Trichrome stained after sacrificing the rats in the end of experiments, so as to investigate the accumulation of collagen in blue portion of cardiocardiomyocytes. The larger the blue area was, the more the accumulation of the collagen was, which represented the more serious heart fibrosis. FIG. 21 showed the Masson Trichrome staining analysis results of rat heart tissues slices of each group. In comparison with sham group, the accumulation of collagen in blue area was significantly increased in DM group; in comparison with DM group, the accumulation of collagen in blue area in treatment group (DM+ADSC group and DM+E-ADSC group) was less. FIG. 22 showed the Western blot analysis of protein expression amount of cardiac fibrosis related proteins. As shown in this figure, in comparison with sham group, the expression level of cardiac fibrosis related proteins was significantly increased in DM group; in comparison with DM group, the expression level of cardiac fibrosis related proteins was significantly decreased in treatment group (DM+ADSC group and DM+E-ADSC group).

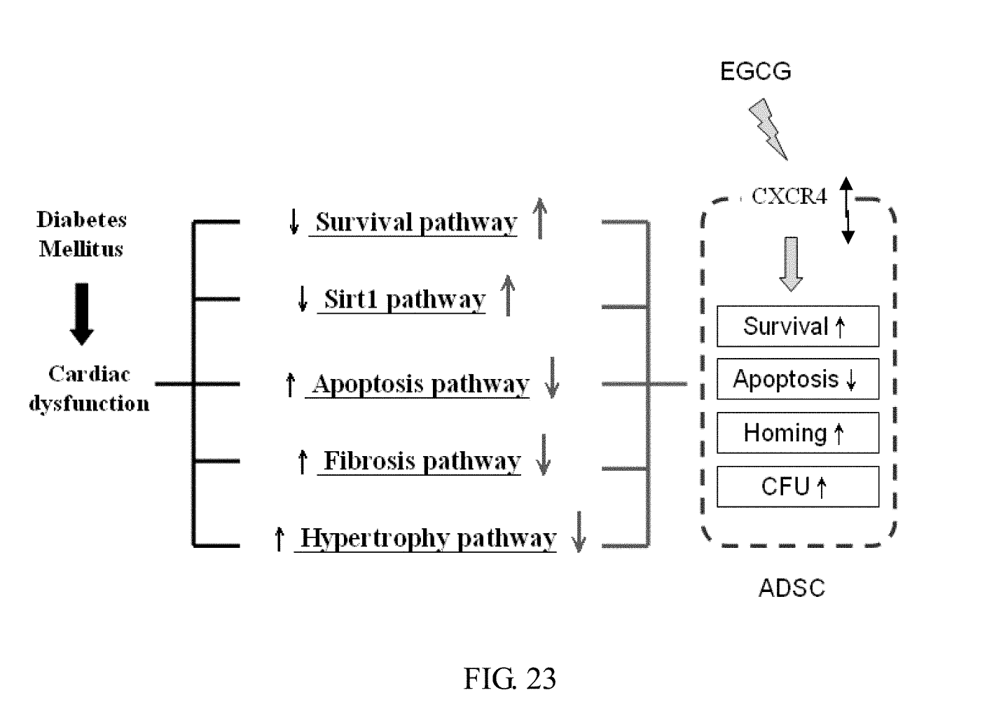

According to the above experiment results, diabetes would cause the rat cardiac tissue damaged, and the adipose-derived stem cells could make the cardiac tissue regenerate from diabetic-induced heart tissue damage. When the adipose-derived stem cells were preconditioned with epigallocatechin gallate (EGCG) of green tea, the regeneration ability of stem cells would be increased significantly from diabetic-induced heart tissue damage. Therefore, the epigallocatechin gallate (EGCG) of green tea precondition would increase the regeneration ability of stem cells. Besides, the epigallocatechin gallate (EGCG) of green tea could increase the expression level of CXCR4 protein on the cell membrane of adipose-derived stem cells. The proliferation ability, survival ability, ability of against apoptosis and migration ability would be increased when the CXCR4 protein expression level was increased on the cell membrane of adipose-derived stem cells. The animal experiment results showed that the cardiomyocytes regenerated from the stem cells perform better function after epigallocatechin gallate (EGCG) of green tea precondition compared with the cardiomyocytes without epigallocatechin gallate (EGCG) of green tea precondition. FIG. 23 showed the mechanism of epigallocatechin gallate (EGCG) of green tea induced regeneration of adipose-derived stem cells to cardiomyocytes through increasing the expression level of CXCR4 protein.

The present invention proved the epigallocatechin gallate (EGCG) of green tea could induce regeneration of adipose-derived stem cells to make the diabetes-induced cardiomyocytes damage recover through increasing the CXCR4 protein expression level. If the epigallocatechin gallate (EGCG) of green tea could be used for treating stem cells clinically, the problem of reinfusion dosage of stem cells would be solved. Therefore, when the treating effect has been evaluated, stem cell therapy could have more significant effect under the limitation of reinfusion dosage of stem cells.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

What is claimed is:

1. A therapeutic compound for repairing cardiac injuries, wherein the therapeutic compound comprises an adipose-derived stem cell.

2. The therapeutic compound of claim 1, wherein the cardiac injuries are caused by diabetes or high-blood glucose.

3. The therapeutic compound of claim 1, wherein the compound further comprises an epigallocatechin gallate (EGCG) of green tea.

4. The therapeutic compound of claim 1, wherein the adipose-derived stem cell and the epigallocatechin gallate (EGCG) of green tea are pretreated for 2 hours.

5. The therapeutic compound of claim 4, wherein the concentration of the epigallocatechin gallate (EGCG) of green tea used for pre-treating the adipose-derived stem cell is 5-15 μM.

6. The therapeutic compound of claim 4, wherein the concentration of the epigallocatechin gallate (EGCG) of green tea used for pre-treating the adipose-derived stem cell is lower than 20 μM.

7. A method for repairing cardiac injuries, which includes administrating a 1×10⁵ adipose-derived stem cells containing therapeutic compound into a subject via intravenous injection.

8. The therapeutic compound of claim 7, wherein the cardiac injuries are caused by diabetes or high-blood glucose.

9. The therapeutic compound of claim 7, wherein the method includes administrating an epigallocatechin gallate (EGCG) of green tea pre-treated 1×10⁵ adipose-derived stem cells into a subject via intravenous injection.

10. The therapeutic compound of claim 7, wherein the adipose-derived stem cell and the epigallocatechin gallate (EGCG) of green tea are pretreated for 2 hours.

11. The therapeutic compound of claim 10, wherein the concentration of the epigallocatechin gallate (EGCG) of green tea used for pre-treating the adipose-derived stem cell is 5-15 μM.

12. The therapeutic compound of claim 10, wherein the concentration of the epigallocatechin gallate (EGCG) of green tea used for pre-treating the adipose-derived stem cell is lower than 20 μM.

13. A method for increasing the carbohydrate tolerance and moving ability of stem cells, wherein the method includes adding an epigallocatechin gallate (EGCG) of green tea into an adipose-derived stem cell culture medium.

14. The therapeutic compound of claim 13, wherein the concentration of the epigallocatechin gallate (EGCG) of green tea is 5-15 μM.

15. The therapeutic compound of claim 13, wherein the method further comprises taking out the adipose-derived stem cells from the epigallocatechin gallate (EGCG) of green tea containing adipose-derived stem cell culture medium after 2 hours culture.
